(19) United States
(12) Patent Application Publication
(10) Pub. No.: US 2007/0005116 A1
(43) Pub. Date: Jan. 4, 2007

(54) Implantable, fully integrated and high performance semiconductor device for retinal prostheses

(75) Inventor: Wai Lo, Lake Oswego, CA (US)

Correspondence Address: LSI LOGIC CORPORATION, 1621 BARBER LANE, MS: D-106, MILPITAS, CA, 95035, US

(73) Assignee: LSI Logic Corporation

(21) Appl. No.: 11/172,486

(22) Filed: Jun. 30, 2005

(51) Int. Cl. A61N 1/00

(52) U.S. Cl. 607/54

(57) ABSTRACT

A retinal prosthesis system which includes an exterior unit that generates electronic images and transmits them to an interior unit that stimulates neurons using electrodes. The interior unit consists of an implantable, fully integrated and high performance device which includes semiconductor devices having much smaller critical dimensions than the prior art, i.e., smaller than 1 μm, e.g., 65 nm, which then enables the incorporation of new functionality as well as the driving of electrode arrays. The electrodes are preferably either nano-tubes or nano-fibers of conducting materials, and their surfaces are engineered so as to achieve appropriate degrees of biocompatibility. Specifically, the nano-tubes and nano-fibers are preferably coated with nano-laminates of either organic or inorganic materials.

### BACKGROUND

The present invention generally relates to devices associated with retinal prostheses, and more specifically-relates to an implantable, fully integrated and high performance semiconductor device for retinal prosthesis.

Tens of millions of people worldwide are blind for various reasons, ranging from birth defects to diseases. Fortunately, it may be possible to restore vision for some of those people, if the cause of their loss of vision falls into certain categories. Among the curable blindness cases are Retinitis Pigmentosa (RP) and Age Related Macular Degeneration (AMD).

RP is a result of a genetic disorder and can be found in 1 of every 4,000 people (i.e., about 1.5 million people worldwide). AMD, on the other hand, is caused by the degeneration of the photoreceptor cells (rods and cones) at the back of the retina over the course of a human life. At least 10% of the U.S. population in the 65-75 age group has lost some central vision due to AMD, and the percentage is certainly higher for those older than 75. This translates to an AMD patient population of at least 5 million in the U.S. alone. This number will increase in the coming years due to the aging of the population as well as longer life expectancy.

To appreciate how medical procedures can restore vision to such patients, it is necessary to review briefly the functioning of a healthy human eye. The retina of a human eye (about 400 μm in thickness) consists of photo-receptor cells (rods and cones) that convert light transmitted through the lens and vitreous medium into electronic pulses. These pulses are then transmitted through various neurons such as the bi-polar cells and ganglion cells, which are typically 10-20 μm in size, across the thickness of the retina before flowing along optic nerves and processed by the brain to generate an image perception. The neurons and optic nerves are not responsive to light so that vision is lost once the photoreceptors are damaged. Both the RP and AMID patients have lost, partially or completely, the photoreceptors, but their neurons are still functional. Hence, it is possible to generate image perceptions to these patients by controlled electrical stimulations with spatial patterns on the ganglion cells of their retina. The present invention is directed to the design and implementation of a retinal prosthesis that can restore the vision of certain blind patients.

There are various versions of retinal prosthesis devices which have been tested on human patients. The most advanced types of devices which have been tested on human patients so far consist of two units as shown in FIG. 1. As shown, the system consists of a first, exterior unit 10 which is positioned outside the eyes, and another, interior unit 12 which is an intraocular implanted unit. The exterior unit 10 captures images using a video camera before processing, encoding and transmitting the images using RF telemetry (i.e., magnetic induction (represented by line 13 in FIG. 1) between an exterial coil 14 and an interial coil 16) to the second unit 12 in the eyes which recovers the transmitted power, decodes the signals and drives an array of electrodes 18 using a flexible cable 20 (see, for example, W. Liu et al., “Dual Unit Retinal Prosthesis”, IEEE EMBS97, which is hereby incorporated herein by reference in its entirety). The electrodes 18 finally stimulate the ganglion cells according to the driving signals and generate a percept.

The designs of these devices are closely related to the special features of human eyes, including constant eye motions, small sizes and delicate retina tissues. The bi-unit nature of the system, for example, is a way to reduce the volume of the intraocular implant, whereas the detaching of the electronic components from the electrodes joined by a cable) inside the eyes is for reducing the stress on the delicate retina. Although the actual structures of the devices may be different, these features need to be taken into account in designing future generations of retinal prosthesis.

As far as the inventor of the current invention knows, the most advanced retinal prosthesis tested on human patients so far included only 25 electrodes. They were therefore designed not to restore complete vision, but to enable certain object recognition capability of the patients. These retinal prosthesis typically used CMOS devices of larger than 1 μm critical dimensions and electrodes of up to 400 μm in diameter, where each electrode stimulates nearly 1000 ganglion cells. Consequently, these devices have poor resolution and limited image processing capability, although both are within the design specifications. As a step forward to improve the performance of retinal prosthesis, an attempt had been made to reduce the electrode size by the fabrication of cylindrical channels about 1 μm in diameter on glass plates and fill the channels with a metal which then gives rise to a large number of electrodes that may improve the resolution of stimulated percepts (see D. Scribner et al., “Intraocular Retinal Prosthesis Test Device”, 23^{rd} Annual Conference of the IEEE Engineering to Medicine and Biology Society, Istanbul, Turkey, October 2001, which is hereby incorporated herein by reference in its entirety). As far as the inventor of the present invention knows, this latter device is yet to receive clinical tests.

Even if the latter device can give high-resolution stimulations, there are still a lot of technical barriers that need to be overcome if a device that fully restores vision is to be developed. These barriers are:
1. Electrode materials: The most commonly used electrode materials are Au and Pt, because of their chemical stability in bulk forms. When used as small electrodes (e.g., 10-400 μm in diameter), however, both exhibit corrosion at the normal stimulation threshold charge density of 0.8-1 mC/cm², which is the minimum electric charge density needed to generate a neuron signal. An emerging electrode material is IrO₂. It can sustain the required stimulation charge without generating toxic by-produces, but tends to have organic film deposits on its surface during operation, which changes the dielectric parameters of the devices gradually. A new electrode material that is chemically stable and mechanically robust needs to be developed.
2. Biocompatibility: Chemically stable and mechanically robust electrode materials, unfortunately, are not always compatible with the biological environment of the eyes. Quoting the examples of Au and IrO₂ again, they either give toxic by-products or encourage the deposition of undesirable substances of the electrode surfaces over time. In some cases, implanted artifacts can also lead to the development of tissues around them. It is hence important to ensure the biocompatibility of any new material used in the implants.
3. System functionality: As mentioned earlier, the systems tested so far were designed to enable pattern recognition capability instead of restoring vision of the patients, which could then compromise on their capacity to process complex images. In addition, the small number of electrodes used inevitably led to the use of relatively simple driving circuits, and are in need of full revision as well as upgrading. Also taking into account the impracticality of replacing the implants due to minor malfunctioning, future prosthesis systems should contain trouble shooting and remnant facilities.
4. Electronic devices: More complex systems functionality together with the limitation on the size of the implants requires the critical dimensions of the electronic devices, e.g., their gate lengths, to be much smaller than the ones tested. With smaller critical dimensions, a large number of devices can be packed onto each implant, the speed of signal processing can be faster and the consumption of power can be reduced.
5. System integration: The intraocular unit of the prosthesis systems tested has separated electronic components and electrodes that are connected by a cable (see FIG. 1). This is for avoiding excessive stress on the delicate retina, but on the other hand loads the interior of the eyes with another object. With smaller electronic devices and smaller volume of the implants, it would be desirable to integrate the electronic components with the electrodes to form a truly system-on-chip implantable prosthesis.

### OBJECTS AND SUMMARY

An object of an embodiment of the present invention is to provide an implantable, fully integrated and high performance semiconductor device for retinal prosthesis.

Another object of an embodiment of the present invention is to provide a semiconductor device for retinal prosthesis which uses large arrays of conducting nano-tubes or nano-fibers with diameters 1.2-100 nm in place of the 1-400 μm conductor electrodes used by the prior art

Yet another object of an embodiment of the present invention is to use electronic devices with critical dimensions substantially smaller than 1 μm, e.g., 65 nm, in place of the larger feature size electronic devices adopted by the previous retina prosthesis implants.

Briefly, and in accordance with at least one of the foregoing objects, an embodiment of the present invention provides a retinal prosthesis system which includes an exterior unit that is configured to generate electronic images and transmit them to an interior unit that stimulates neurons using electrodes. The interior unit consists of electronic devices of very small critical dimensions which enables the incorporation of new functionality as well as the driving of electrode arrays. The electrodes are,either nano-tubes or nano-fibers of conducting materials, and their surfaces are engineered so as to achieve appropriate degrees of biocompatibility.

The electrodes are preferably either nano-tubes or nano-fibers of conducting materials, and their surfaces are engineered so as to achieve appropriate degrees of biocompatibility. Specifically, the nano-tubes and nano-fibers are preferably coated with nano-laminates of either organic or inorganic materials.

### BRIEF DESCRIPTION OF THE DRAWINGS

The organization and manner of the structure and operation of the invention, together with further objects and advantages thereof, may best be understood by reference to the following description, taken in connection with the accompanying drawing, wherein:

FIG. 1 is a schematic diagram which illustrates the structures of a conventional system for restoring vision to a damaged retina;

FIG. 2 is a schematic diagram which illustrates the structures of an intraocular retinal prosthesis which is in accordance with an embodiment of the present invention; and

FIG. 3 provides a more detailed circuit block diagram of the exterior and interior units shown in FIG. 2.

### DESCRIPTION

While the invention may be susceptible to embodiment in different forms, there are shown in the drawings, and herein will be described in detail, specific embodiments of the invention. The present disclosure is to be considered an example of the principles of the invention, and is not intended to limit the invention to that which is illustrated and described herein.

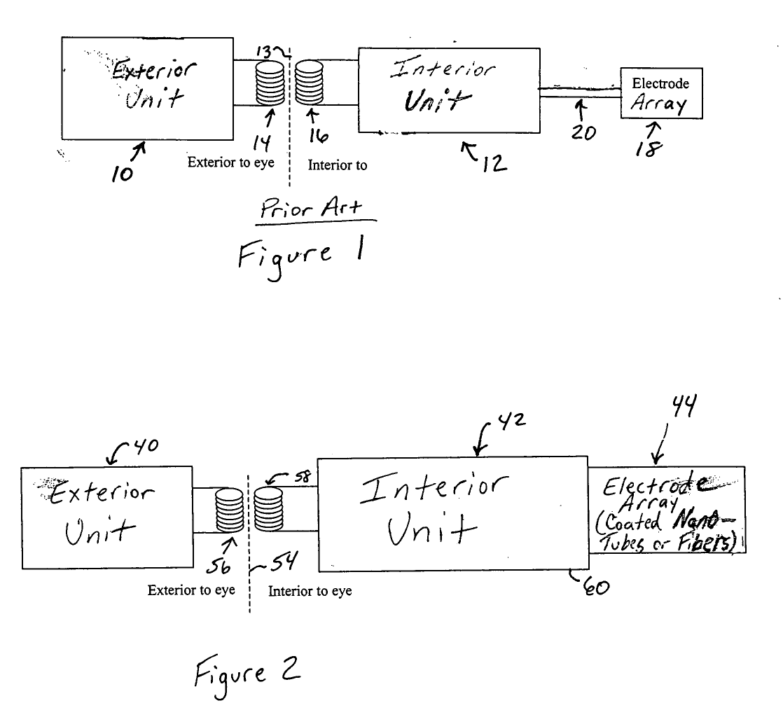

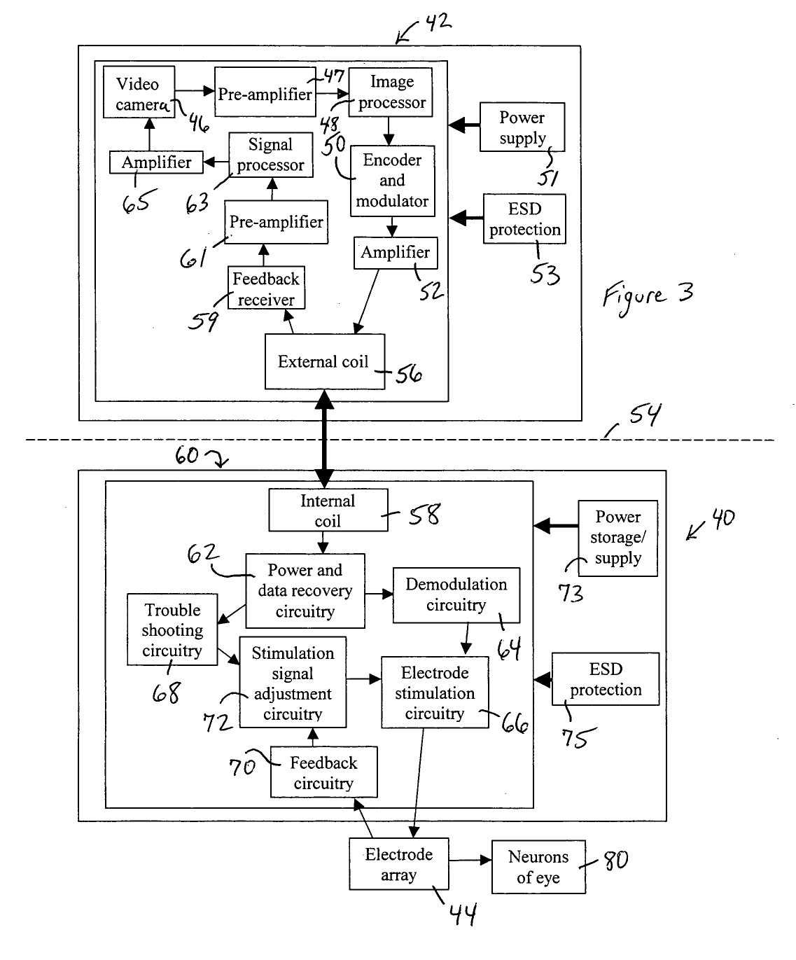

FIG. 2 illustrates the architectural features of an intraocular retina prosthesis device which is in accordance with an embodiment of the present invention. Similar to the previous devices, it has an exterior unit 40 that generates electronic images and transmits them to an interior unit 42 that stimulates neurons 80 using electrodes 44. Specifically, similar to the previous devices, as shown in FIG. 3, the exterior unit 40 may consist of a video camera 46, pre-amplifier 47, image processor 48, encoder and modulator 50, power supply 51, ESD protection unit 53 and amplifier 52, all of which are collectively configured to video the outside world, generate electronic images based on what is “viewed” by the video camera 46, and transmit the electronic images to the interior unit 42 preferably via RF telemetry (i.e., magnetic induction (represented by line 54 in FIG. 2) between an exterial coil 56 and an interial coil.58, although data can also be transmitted through other wireless means. Preferably, the exterior unit 42 is also configured to process feedback (via a feedback unit 59) received from the interior unit 40, and also includes a pre-amplifier 61, signal processor 63, and amplifier 65.

The interior unit, however, consists of an implantable, fully integrated and high performance device which includes semiconductor devices on an electronic chip 60. The semiconductor devices have much smaller critical dimensions than the prior art, i.e., smaller than 1 μm, e.g., 65 nm, which then enables the incorporation of new functionality as well as the driving of electrode arrays. Preferably, as shown in FIG. 3, the electronic chip 60 consists of small critical dimension electronic devices for power and data recovery 62, demodulation 64, electrode stimulation 66, trouble shooting facility 68, feedback circuitry 70 for feeding back information to the exterior unit 40, stimulation signal adjustment circuitry 72, power storage/supply unit 73 and ESD protection unit 75.

The electrodes 44 are preferably either nano-tubes or nano-fibers of conducting materials which are disposed on the electronic chip, connected to the electronic devices, and their surfaces are engineered so as to achieve appropriate degrees of biocompatibility. Specifically, the nano-tubes and nano-fibers are preferably coated with nano-laminates of either organic or inorganic materials.

Features of the present invention include:
(a) Electrodes: Large arrays of conducting nano-tubes or nano-fibers with diameters 1.2-100 nm are used in place of the 1-400 μm conductor electrodes by the prior art (see D. Scribner et al., “Intraocular Retinal Prosthesis Test Device”, 23^{rd} Annual Conference of the IEEE Engineering to Medicine and Biology Society, Istanbul, Turkey, October 2001). These nano-tubes or nano-fibers can either be grown directly on substrates from precursors such as carbon (i.e., forming carbon nano-tubes/nano-fibers) or incorporated from existing nano-tubes/nano-fibers by different techniques such as spin coating and etching. These nano-tubes/nano-fibers can either form nano-channels on the surface of the retina or be normal to the retina surface so that neurons are stimulated by their tips.
(b) Engineered biocompatibility: The surface of nano-tubes and nano-fibers can be functionalized in order to tailor the compatibility with the ocular environment of the patients. This can be achieved by coating the nano-tubes and nano-fibers with nano-laminates of either organic or inorganic materials. Coating techniques available include plasma assisted polymer coating (see, for example, P. He at al., “Plasma coating and enhanced dispersion of carbon nanotubes”, preprint; D. Shi et al., “Plasma deposition of ultrathin polymer films on carbon nanotubes”, Appl. Phys. Lett. 81 (2002) 1; and D. Shi et al., “Plasma coating of carbon nanofibers for enhanced dispersion and interfacial bonding in polymer composites”, Appl. Phys. Lett. 83 (2003) 1, all three of which are incorporated herein by reference in their entirety), sol-gel coating and atomic layer deposition.
(c) Electronic devices: Electronic devices with critical dimensions substantially smaller than 1 μm, e.g., 65 nm, will be used in place of the larger feature size electronic devices of adopted by the previous retina prosthesis implants. This can be achieved by using state-of-the-art process technology, and further shrinking of the devices will be possible in the coming years. Smaller electronic devices can increase their packing density by a factor of 10³ currently and up to a factor of 10⁴ in a decade. This will enable the incorporation of more functionality into the intraocular system as well as enhancing its performance.
(d) System functionality: With the large number of electronic devices as well as electrodes available, more functionality such as troubleshooting, tuned electrode driving and programmed image processing can be realized. Tuned electrode driving is needed in case some of the electrodes either will cease functioning or have degraded electric properties over time, due to various reasons. Programmed image processing will allow the optimization of image qualities based on the conditions of the electronic devices.
(e) System integration: Smaller electronic devices and lighter chip will enable the integration of the chip with the electrode arrays, without overloading the retina. This on one hand can reduce stress on the ocular environment by removing the connection cable between the chip and the electrodes, which could only become bulkier with higher number of electrodes. With the convenience of back-end metal level programmability of platform ASIC (e.g. Rapid Chip) and the maturity of chip level packing technologies, the integration between these two modules can be carried out with at metal connection level of chip packaging level.

There are a lot of advantages of the present system, mainly brought about by the use of new electrode materials and small feature electronic devices, as well as the incorporation of new functionality. They are:
(i) Lighter electronic chip: This has been mentioned hereinabove and is the direct product of using smaller electronic devices. A lighter electronic chip leads to the introduction of more functionality, integration with electrodes and better performance of the system.
(ii) Higher resolution of images: The use of nanometer size electrodes in large numbers enabled the stimulation of individual neuron cells, which is to be contrasted with the stimulation of groups of cells in the previous systems, and can hence increase the resolution of percepts when a good quality image is transmitted to the electrode drivers. It can also reduce the unintended stimulation of neurons through their fibers by nearby electrodes, which will lead to higher signal-to-noise ratios of the signals or higher clarity of percepts.
(iii) Lower threshold charge density: Studies showed that the threshold charge density and voltage (charge density or voltage needed for inducing a neuron signal) are inversely proportional to the distance between two electrodes in the bi-polar electrode arrangement (see S. K. Kelly, “A System for Efficient Neural Stimulation with Energy Recovery”, Ph. D Thesis, MIT, 2003, which is the electrode design in the most advanced retina prosthesis. With high density of electrodes and hence reduced electrode spacing, the threshold charge density and voltage can be selected for optimizing the operation conditions and the quality of the percepts of the system. This not only can improve the performance of the system, but can also reduce power consumption by the system.
(iv) More reliable system: The large number of electrodes, troubleshooting circuitry and programmable stimulations can all lead to a more reliable system. For example, since each neuron will be stimulated by more than one electrode (the diameter is 1.2-100 nm and size of a ganglion cell is 10-20 μm, i.e., 10²-10³, larger than an electrode), there will be a lot of redundant electrode per neuron and the failure of some of these electrodes will not hinder the functioning of the system, if tuned stimulation functionality is incorporated in the system. The failure or degradation of any electrode in the previous systems, however, cannot be repaired except by replacing the retina prosthesis. Higher reliability of the system understandably also reduces the need for replacing the prosthesis through surgical process and hence improves the quality of life of the patients.
(v) Better biocompatibility: The coating of the electrodes effectively forms a composite material with almost unlimited possibility in synergetic combinations of electrodes and coats. It opens a whole new route for engineering the biocompatibility and enhancing chances of success, or even fine-tuning the properties according to the conditions of the patients. This is certainly more superior to the existing electrodes which have biocompatibility issues.

As such, an embodiment of the present invention provides an exterior unit as well as an implantable, fully integrated and high performance semiconductor device for retinal prosthesis.

While embodiments of the present invention are shown and described, it is envisioned that those skilled in the art may devise various modifications of the present invention without departing from the spirit and scope of the appended claims.

What is claimed is:

1. A retinal prosthesis system comprising: a first unit configured to be implanted in an eye, said first unit comprising an electronic chip having semiconductor devices thereon and nano-tubes or nano-fibers of conducting materials connected to the semiconductor devices; and a second unit which is configured to be external to the eye and which is configured to generate electronic images and transmit said electronic images to the first unit, said first unit configured to receive the electronic images and stimulate neurons of the eye using the electrodes, wherein the electrodes comprise at least one of nano-tubes and nano-fibers.

2. A retinal prosthesis system as recited in claim 1, wherein surfaces of the nano-tubes or nano-fibers are coated with nano-laminates.

3. A retinal prosthesis system as recited in claim 1, wherein surfaces of the nano-tubes or nano-fibers are coated by at least one of: plasma assisted polymer coating, sol-gel coating and atomic layer deposition.

4. A retinal prosthesis system as recited in claim 2, wherein the surfaces of the nano-tubes or nano-fibers are coated with nano-laminates of either organic or inorganic materials.

5. A retinal prosthesis system as recited in claim 1, wherein the semiconductor devices on the electronic chip consists of devices having critical dimensions smaller than 1 μm.

6. A retinal prosthesis system as recited in claim 1, wherein the semiconductor devices on the electronic chip consists of devices having critical dimensions of 65 nm.

7. A retinal prosthesis system as recited in claim 1, wherein each of the nano-tubes or nano-fibers has a diameter 1.2-100 nm.

8. A retinal prosthesis system as recited in claim 1, wherein the nano-tubes or nano-fibers are at least one of: grown directly on the electronic chip, spin coated on the electronic chip, or etched onto the electronic chip.

9. A retinal prosthesis system as recited in claim 1, wherein the nano-tubes or nano-fibers form nano-channels on a surface of a retina of the eye.

10. A retinal prosthesis system as recited in claim 1, wherein the nano-tubes or nano-fibers are normal to a retina surface of the eye such that neurons are stimulated by tips thereof.

11. An implantable device for retinal prosthesis comprising: an electronic chip having semiconductor devices thereon and electrodes connected to the semiconductor devices, said electrodes comprising nano-tubes or nano-fibers, said electronic chip configured to receive electronic images from a device external to the eye and stimulate neurons of the eye using the electrodes.

12. An implantable device for retinal prosthesis as recited in claim 11, wherein surfaces of the nano-tubes or nano-fibers are coated with nano-laminates.

13. An implantable device for retinal prosthesis as recited in claim 11, wherein surfaces of the nano-tubes or nano-fibers are coated by at least one of: plasma assisted polymer coating, sol-gel coating and atomic layer deposition.

14. An implantable device for retinal prosthesis retinal prosthesis system as recited in claim 12, wherein the surfaces of the nano-tubes or nano-fibers are coated with nano-laminates of either organic or inorganic materials.

15. An implantable device for retinal prosthesis as recited in claim 11, wherein the semiconductor devices on the electronic chip consists of devices having critical dimensions smaller than 1 Jim.

16. An implantable device for retinal prosthesis as recited in claim 11, wherein the semiconductor devices on the electronic chip consists of devices having critical dimensions of 65 nm.

17. An implantable device for retinal prosthesis as recited in claim 11, wherein each of the nano-tubes or nano-fibers has a diameter 1.2-100 nm.

18. An implantable device for retinal prosthesis as recited in claim 11, wherein the nano-tubes or nano-fibers are at least one of: grown directly on the electronic chip, spin coated on the electronic chip, or etched onto the electronic chip.

19. An implantable device for retinal prosthesis as recited in claim 11, wherein the nano-tubes or nano-fibers form nano-channels on a surface of a retina of the eye.

20. An implantable device for retinal prosthesis as recited in claim 11, wherein the nano-tubes or nano-fibers are normal to a retina surface of the eye such that neurons are stimulated by tips thereof.
